(19) United States
(12) Patent Application Publication
(10) Pub. No.: US 2014/0206970 A1
(43) Pub. Date: Jul. 24, 2014

(54) EVALUATION AND DISPLAY OF GLUCOSE DATA

(71) Applicant: Park Nicollet Institute, St. Louis Park, MN (US)

(72) Inventors: David M. Wesley, Hudson, WI (US); Richard M. Bergenstal, Plymouth, MN (US)

(73) Assignee: Park Nicollet Institute, St. Louis Park, MN (US)

(21) Appl. No.: 14/161,031

(22) Filed: Jan. 22, 2014

Related U.S. Application Data
(60) Provisional application No. 61/755,406, filed on Jan. 22, 2013.
(60) Provisional application No. 61/769,747, filed on Feb. 26, 2013.

(51) Int. Cl. A61B 5/00; A61B 5/145

(52) U.S. Cl. CPC A61B 5/742; A61B 5/14532
USPC 600/365

(57) ABSTRACT

A glucose evaluation system operates to evaluate and display glucose data. The glucose data is evaluated and a display is generated that presents the glucose data in a form that quickly conveys key information to a caregiver, without requiring the caregiver to spend a great deal of time studying the data or the display.

### CROSS-REFERENCE TO RELATED APPLICATIONS

This application claims priority to U.S. Provisional Application Ser. No. 61/755,406, filed on Jan. 22, 2013, titled “EVALUATION AND DISPLAY OF GLUCOSE DATA;” and to U.S. Provisional Application No. 61/769,747, filed on Feb. 26, 2013, titled “EVALUATION AND DISPLAY OF GLUCOSE DATA,” the disclosures of which are hereby incorporated by reference in their entireties. To the extent appropriate, a claim of priority is made to each of the above disclosed applications.

### BACKGROUND

The measurement and monitoring of blood glucose levels is particularly important to the care and management of diabetes. The most common method of measuring blood glucose is by piercing the skin and applying a small amount of blood to a test strip that is inserted into a glucose monitor. The glucose monitor interrogates the sample and determines the glucose level. Some glucose monitors store the glucose levels as glucose data for subsequent display or transmission.

Continuous glucose monitors have also been developed. Such monitors typically include a disposable glucose sensor that can be inserted under the skin. The continuous glucose monitor performs an interrogation regularly and periodically over an extended period of time, which provides much more data regarding the fluctuations in glucose levels over that time.

### SUMMARY

In general terms, this disclosure is directed to evaluation and display of glucose data. In one possible configuration and by non-limiting example, the glucose data is evaluated and a display is generated that presents the glucose data in a form that quickly conveys key information to a caregiver, without requiring the caregiver to spend a great deal of time studying the data or the display.

One aspect is a method of evaluating and displaying glucose data, the method comprising: receiving at a computing device glucose data for a patient, the glucose data containing data generated by a continuous glucose monitor associated with the patient; and generating a graphical display of the glucose data with the computing device, the graphical display including at least a glucose profile for a modal day, the glucose profile graphically depicting therein: a target range for the glucose data for the patient, including at least an upper boundary and a lower boundary; and a line representing a median value of the glucose data across the modal day.

Another aspect is a method of graphically displaying glucose data, the method comprising: evaluating glucose data, the glucose data including data obtained from a glucose monitor device; generating with a computing device a glucose statistics window based on the evaluation of the glucose data, the glucose statistics window including at least a glucose exposure statistic, a glucose variability statistic, glucose ranges, and a data sufficiency statistic; generating an ambulatory glucose profile window, the ambulatory glucose profile window including a graphical display of the glucose data across a modal day; and generating a daily glucose profile window, the daily glucose profile window including a graphical display of the glucose data corresponding to days of a week.

A further aspect is a glucose data evaluation server, comprising: a computing device; and at least one computer readable storage device, the at least one computer readable storage device storing (i) glucose data based at least in part upon data obtained by a continuous glucose monitor device, and (ii) program instructions, the program instructions being executable by the computing device to: generate a graphical display of the glucose data, the graphical display including at least a glucose profile for a modal day, the glucose profile graphically depicting therein: a target range for the glucose data for the patient, including at least an upper boundary and a lower boundary; and a line representing a median value of the glucose data across the modal day.

A glucose data evaluation server, comprising: a computing device; and at least one computer readable storage device, the at least one computer readable storage device storing (i) glucose data based at least in part upon data obtained by a continuous glucose monitor device, and (ii) program instructions, the program instructions being executable by the computing device to: generate a glucose statistics window based on the glucose data, the glucose statistics window including at least a glucose exposure statistic, a glucose variability statistic, glucose ranges, and a data sufficiency statistic; generate an ambulatory glucose profile window, the ambulatory glucose profile window including a graphical display of the glucose data across a modal day; and generate a daily glucose profile window, the daily glucose profile window including a graphical display of the glucose data corresponding to days of a week.

### BRIEF DESCRIPTION OF THE DRAWINGS

FIG. 1 is a schematic block diagram of an example glucose data evaluation system.

FIG. 2 is a flow chart illustrating an example method of evaluating glucose data.

FIG. 3 is a chart showing an example of raw data received by the glucose data evaluation server of the system.

FIG. 4 is a schematic block diagram illustrating an example database of the glucose data evaluation server.

FIG. 5 is an example block diagram illustrating a glucose data display.

FIG. 6 is a schematic block diagram illustrating an example of the glucose data display engine.

FIG. 7 is an example diagram illustrating a glucose statistics window of the glucose data display described in FIG. 5.

FIG. 8 is an example diagram illustrating an expanded glucose statistics window of the glucose data display described in FIG. 5.

FIG. 9 is an example diagram illustrating an ambulatory glucose profile window of the glucose data display described in FIGS. 5 and 9.

FIG. 10 is an alternative example diagram illustrating an ambulatory glucose profile window of the glucose data display described in FIG. 5.

FIG. 11 is an alternative example embodiment of an AGP window 1100 including CGM data lines, CGM data points, and SMBG data points.

FIG. 12 is an alternative example embodiment of an AGP window 1200 including CGM data lines and CGM data points.

FIG. 13 is an alternative example embodiment of an AGP window 1300 including CGM data lines and SMBG data points.

FIG. 14 is an alternative example embodiment of an AGP window 1400 including CGM data points and SMBG data points.

FIG. 15 is an alternative example embodiment of an AGP window 1500 including only CGM data points.

FIG. 16 is an example diagram illustrating an insulin pump graph window of the glucose data display described in FIG. 5.

FIG. 17 is an example diagram illustrating a daily glucose profile window of the glucose data display described in FIG. 5.

FIG. 18 is a block diagram illustrating an example architecture of a computing device, which can be used to implement various aspects of the system illustrated in FIG. 1.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

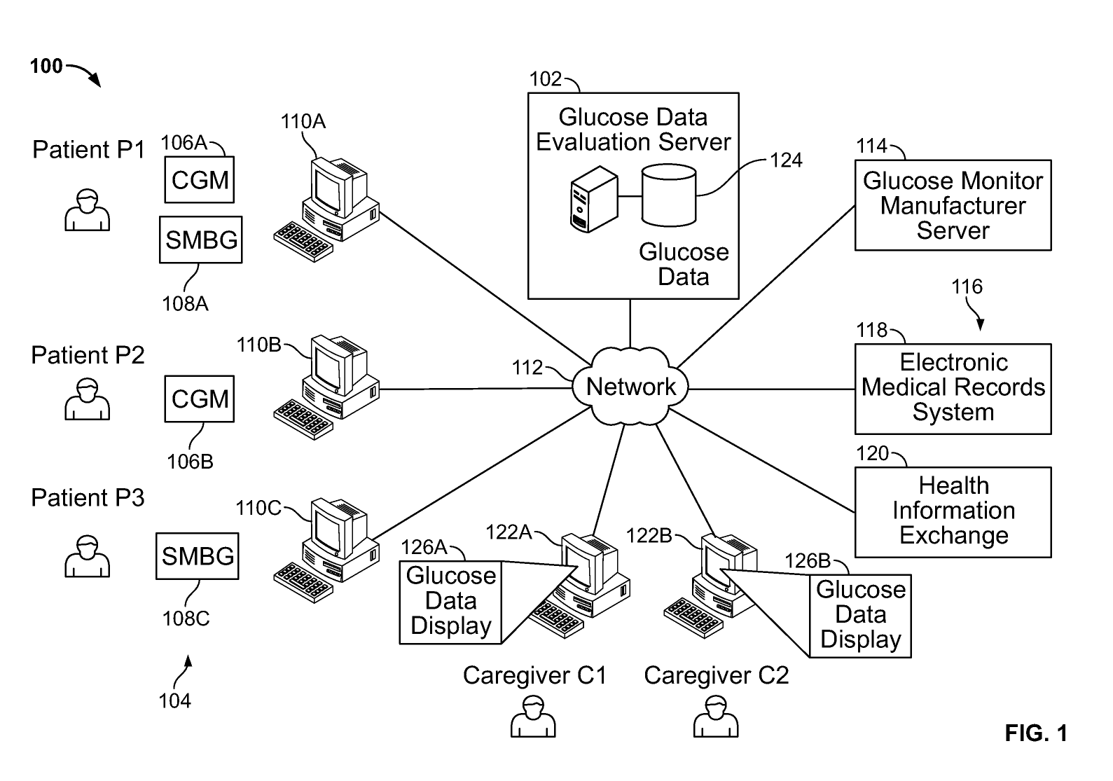

FIG. 1 is a schematic block diagram of an example glucose data evaluation system 100. In the example, the system 100 includes a glucose data evaluation server 102, glucose monitor devices 104, patient computing devices 110, caregiver computing devices 108, glucose monitor manufacturer server 114, and records systems 112.

Multiple patients P (including patients P1, P2, and P3) interact with the glucose data evaluation system 100, which operates to monitor and evaluate the glucose levels of the patients P. In this example, patient P1 has two glucose monitor devices 104, including a continuous glucose monitor (CGM) device 106A and a self-monitoring blood glucose (SMBG) device 108A; patient P2 has a single glucose monitor device 104, such as a CGM device 106B; and patient P3 has a single glucose monitor device 104, such as a SMBG device 108C.

The glucose monitor devices 104 operate to take measurements of the patient's blood glucose level, and save the measurements as glucose data in a computer readable storage device of the glucose monitor devices 104. The CGM devices 106 operate to automatically take glucose measurements periodically and frequently throughout the day, and do not require action by the patient P to obtain the measurements. In contrast, the SMBG devices 108 typically require that a blood sample be obtained and provided onto a test strip by the patient, and therefore the glucose measurements are typically obtained less frequently with the SMBG devices 108 than with the CGM devices 106.

In some embodiments, glucose data from the glucose monitor devices 104 is transferred to a computing device 110 (including computing devices 110A, 110B, and 110C). The computing device 110 can be a desktop or mobile computing device (such as a laptop, smartphone, tablet computer, and the like) or can be another computing device, such as a bedside monitor, for example. Communication between the glucose monitor devices 104 and the computing device 110 can occur through a wired connection, or through a wireless connection, such as using radio frequency communication devices.

In some embodiments, the glucose data from the glucose monitor devices 104 is then transferred across a data communication network from the computing device 110 to another computing device. It is also possible for the glucose data to be transferred from the glucose monitor devices 104 to other computing devices using other data communication techniques. For example, in some embodiments the glucose monitor devices 104 include a cellular data communication device that permits the data to be communicated directly from the glucose monitor devices 104 across a cellular data communication network. In another possible embodiment, data communication can occur across a telephone network, such as by generating and providing audible signals to a telephone with the glucose monitor device 104. Other embodiments utilize other forms of data communication.

The glucose data can be communicated to a variety of possible locations. In one example, the glucose data is transferred to a server 114 operated by the glucose monitor device 104 manufacturer. In another possible embodiment, the glucose data is transferred to one or more of: a records system 116 (such as an electronic medical records system 118 or a health information exchange 120), a caregiver computing device 122, and a glucose data evaluation server 102.

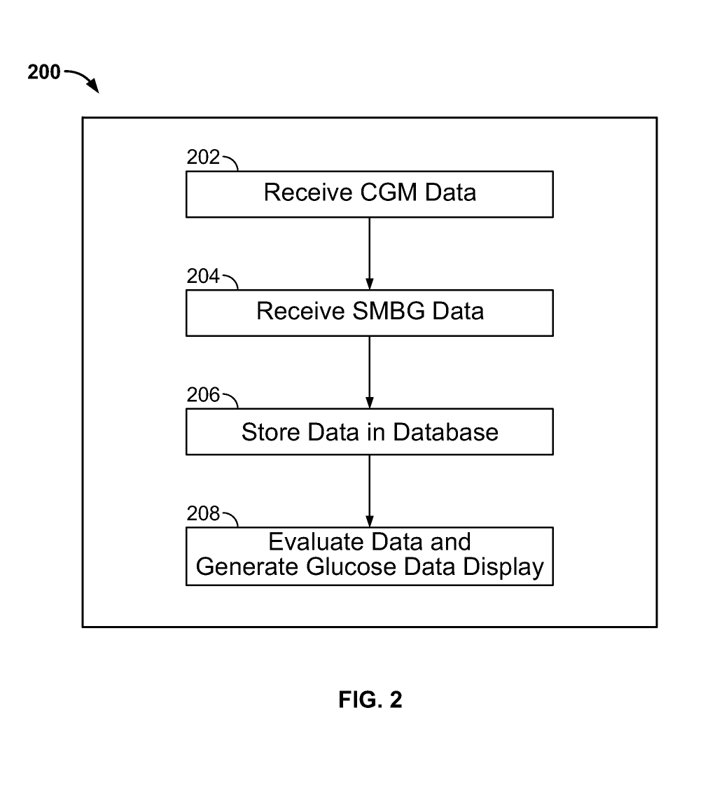

The glucose data is ultimately transferred to the glucose data evaluation server 102. For example, in some embodiments the glucose data is transferred directly from the computing device 110 to the glucose data evaluation server 102. In other embodiments, the data is transferred from another computing device (e.g., server 114, records system 116, or a caregiver computing device 122) to the glucose data evaluation server. Aspects of the glucose data evaluation server 102 are illustrated and described in more detail with reference to FIGS. 2-6 and 18.

It should be noted, however, that in some embodiments the glucose data evaluation server 102 is part of or the same as one of the other computing devices described herein, such as the glucose monitor manufacturer server 114, the electronic medical records system 118, the health information exchange 120, for example. In such cases, further transfer of the glucose data may not be necessary.

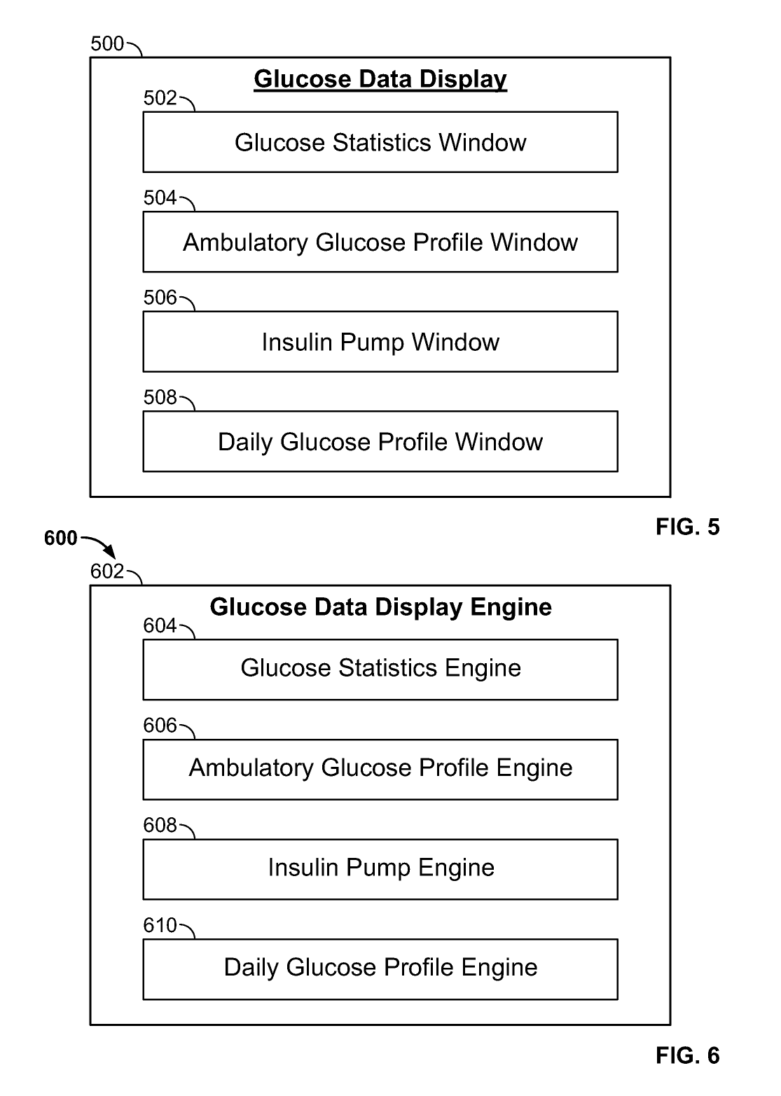

The glucose data is processed and saved in a database 124 accessible to the glucose data evaluation server 102. The glucose data evaluation server 102 then processes the data as described herein, and generates a glucose data display 126. The glucose data display is presented to a caregiver C (such as caregiver C1 or caregiver C2) on a caregiver computing device 122 (computing devices 122A or 122B). The glucose data display presents the data in such a way that the caregiver can quickly and easily understand various information relating to the glucose levels of the patient P (P1, P2, or P3) over a period of time. Examples of the glucose data display 126 are illustrated and described in more detail herein with reference to FIGS. 5 and 7-17.

FIG. 2 is a flow chart illustrating an example method 200 of evaluating glucose data. In this example, the method 200 includes operations 202, 204, 206, and 208.

Operation 202 is performed to receive continuous glucose monitor data from a patient's CGM device 106.

Operation 204 is performed to receive self-monitoring blood glucose data from a patient's SMBG device 108.

Operation 206 is performed to store data received from a patient's CGM device 106 and/or a patient's SMBG device 108.

Operation 208 is performed to evaluate the glucose data and generate a glucose data display.

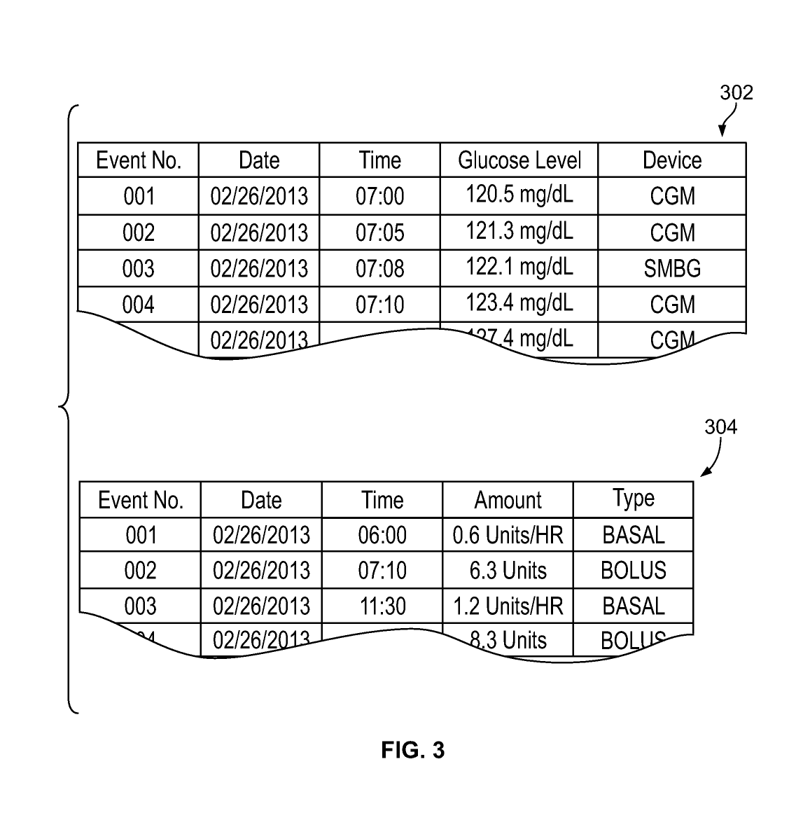

FIG. 3 is a chart showing an example of raw data received by the glucose data evaluation server 102. In this example embodiment, FIG. 3 illustrates an example of raw glucose data 302 and raw insulin pump data 304 received from a patient's P CGM device or a SMBG device. Examples of raw glucose data 302 received are glucose levels (in mg/dL), the date on which the glucose level was received, and the time in which the glucose level was received. Examples of raw insulin pump data 304 received are the amount of insulin received by the patient P (in units), the date on which the insulin was received, and the time in which the insulin was received. In another example embodiment, the type of insulin the patient P received is also received by the server 102 as raw insulin pump data 304. In some embodiments, a patient P receives one or more of basal insulin and bolus insulin.

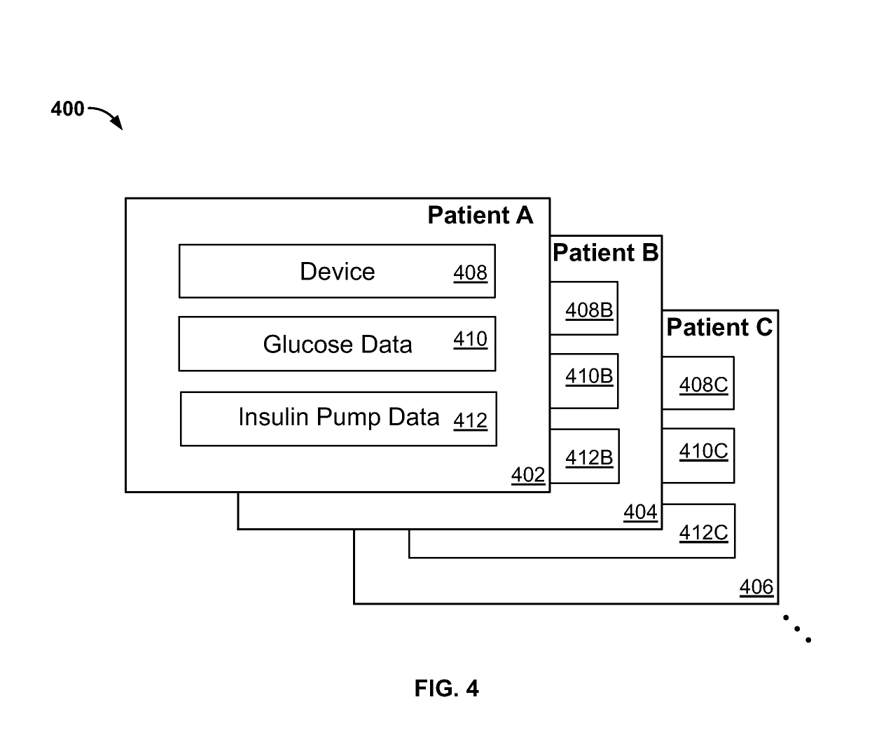

FIG. 4 is a schematic block diagram illustrating an example database 124 (FIG. 1). In this example, the database 400 includes a plurality of patient tables 402, 404, and 406. In this embodiment, table 402 represents information about a patient's P glucose data 408 and insulin data 410 received from a device 412. In some embodiments, a device is a CGM device. In other embodiments, a device is a SMBG device. Similar data is stored in other patient tables 404 and 406, for example. Other embodiments include more or less data than shown in this example.

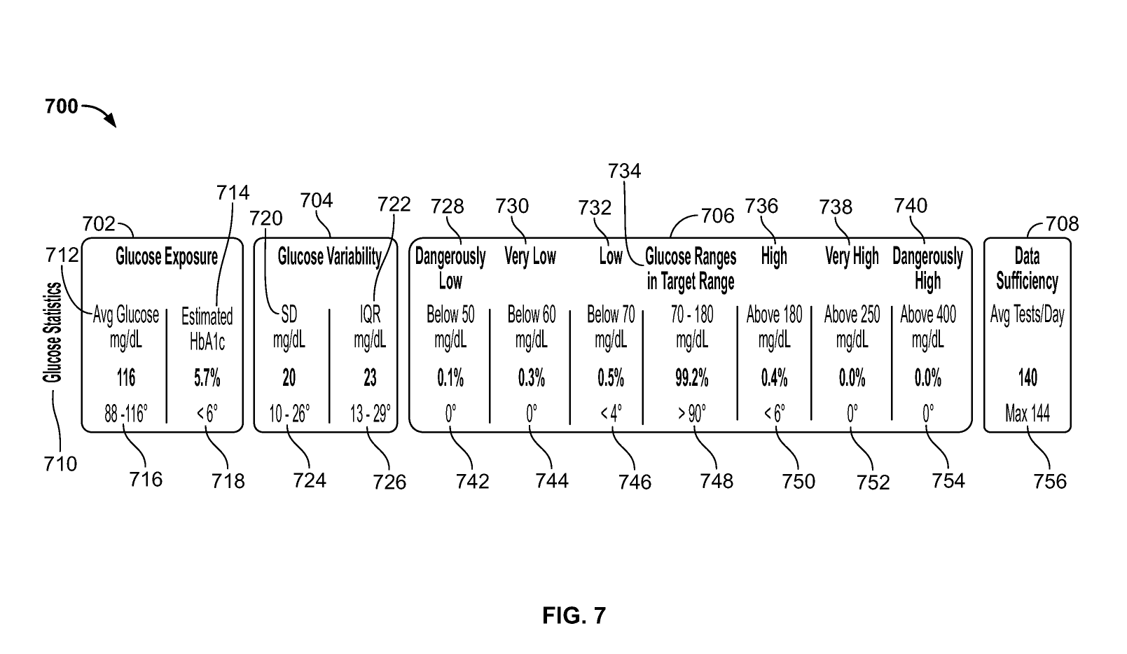

FIG. 5 is an example block diagram illustrating a glucose data display 500. In this example embodiment, the glucose data display 500 includes a glucose statistics window 502, an ambulatory glucose profile window 504, an insulin pump window 506, and a daily glucose profile window 508. In other embodiments, the glucose data display 500 is arranged in other configurations. The glucose data display 500 is discussed in more detail with reference to FIGS. 7-15.

FIG. 6 is a schematic block diagram illustrating an example of the glucose data display engine 602. In some embodiments, the glucose data display engine 602 includes a glucose statistics engine 604, an ambulatory glucose profile engine 606, an insulin pump engine 608, and a daily glucose profile engine 610. Each engine is responsible for calculating and displaying various data displayed on the glucose data display. The glucose data display engine 602 is also responsible for displaying identifying information about a patient P such as the patient's P name, the date range in which the display is compiled, and the total number of tests taken from the patient's P CGM device or SMBG device. In other embodiments, the identifying information further consists of information such as, but not limited to, a patient's P birth date, age, gender, weight, ethnicity, type of diabetes, and date of onset.

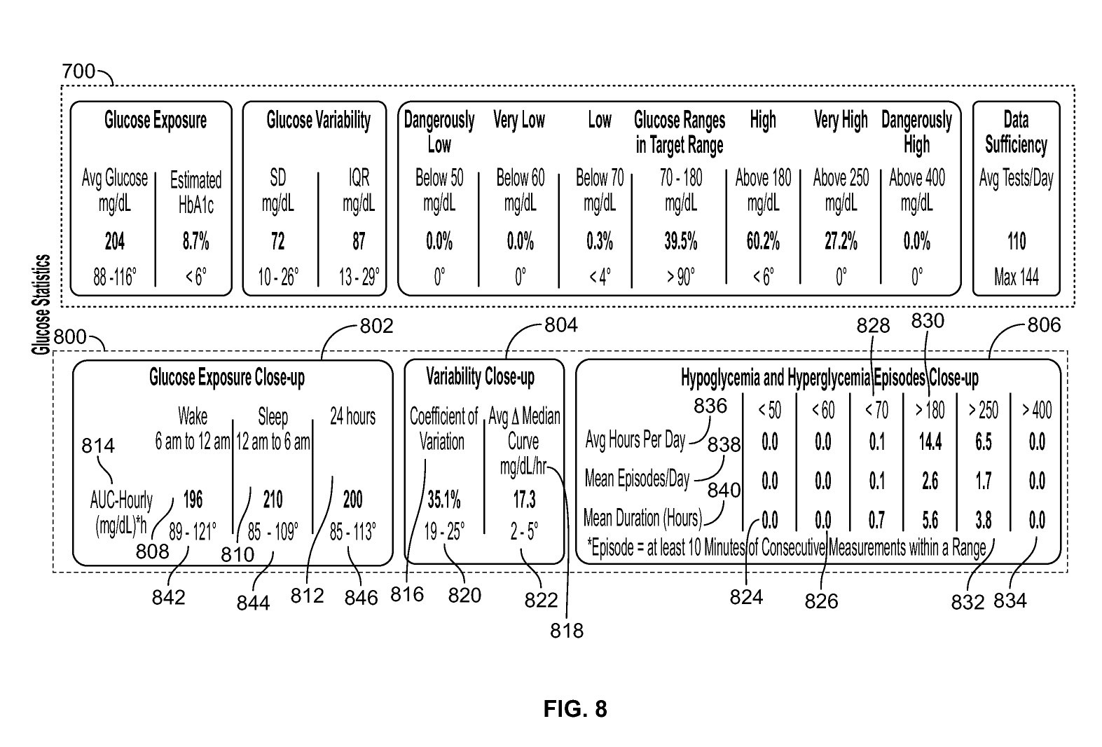

In some embodiments, the glucose statistics engine 604 calculates the patient's P blood glucose measurements including glucose exposure, glucose variability, glucose ranges, and data sufficiency. In other embodiments, other statistics are calculated and displayed. The default unit of measurement for the blood glucose measurement is mg/L. In other embodiments, the blood glucose unit of measurement is displayed in mmol/L. The glucose statistics window is described in more detail with reference to FIGS. 7 and 8.

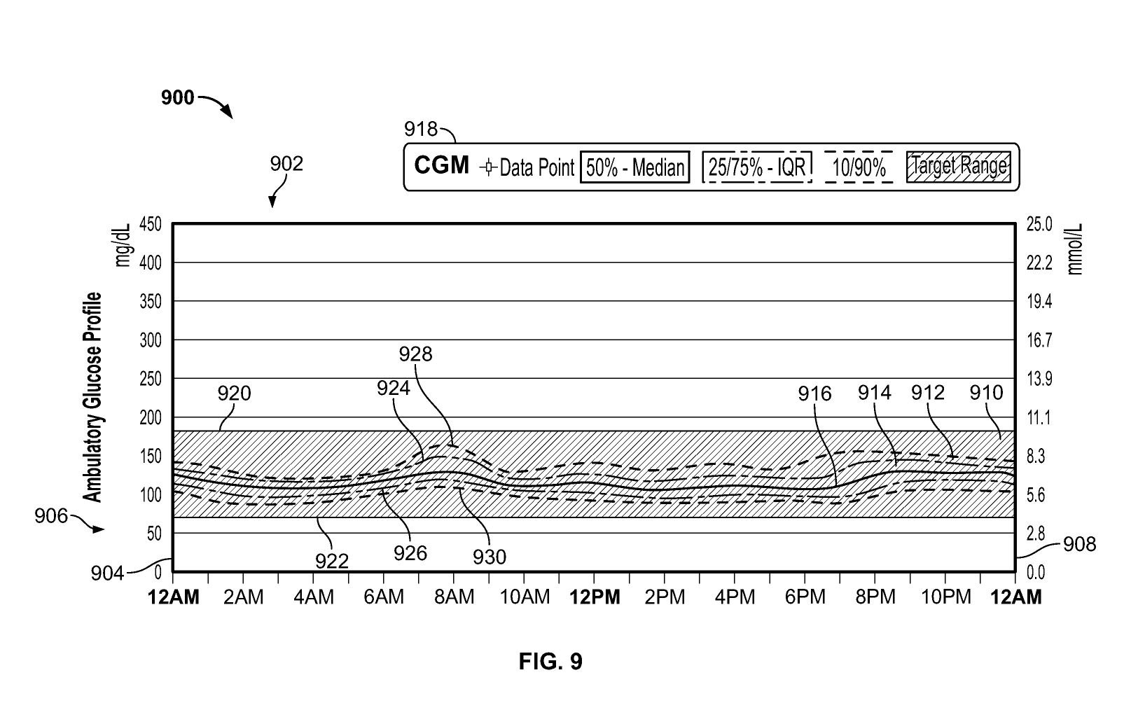

In some embodiments, the ambulatory glucose profile engine 606 calculates and displays a graph describing the patient's P median glucose levels over a 24-hour period. The ambulatory glucose profile engine 606 also displays a default target range, the device from which the glucose data is retrieved, and percentile ranges. The ambulatory glucose profile window is described in more detail with reference to FIGS. 9-13.

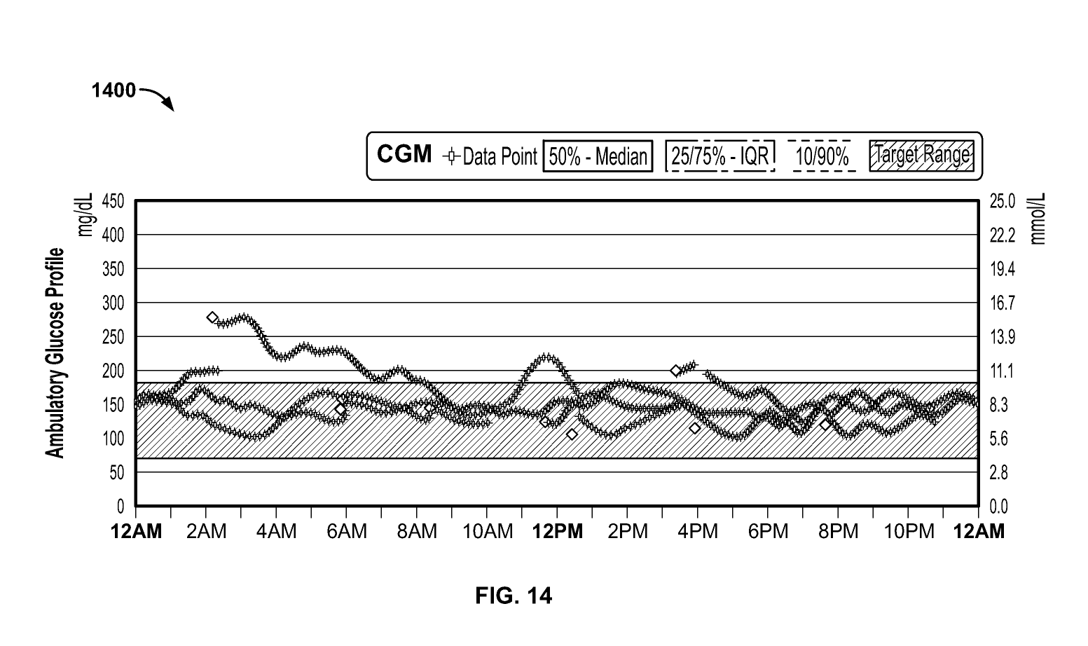

In some embodiments the insulin pump engine 608 calculates and displays the patient's P insulin pump data that is consistent with the ambulatory glucose profile window. In some embodiments, the insulin pump engine 608 calculates and displays both the bolus insulin and basal insulin rates. The insulin pump window is described in more detail with reference to FIG. 14.

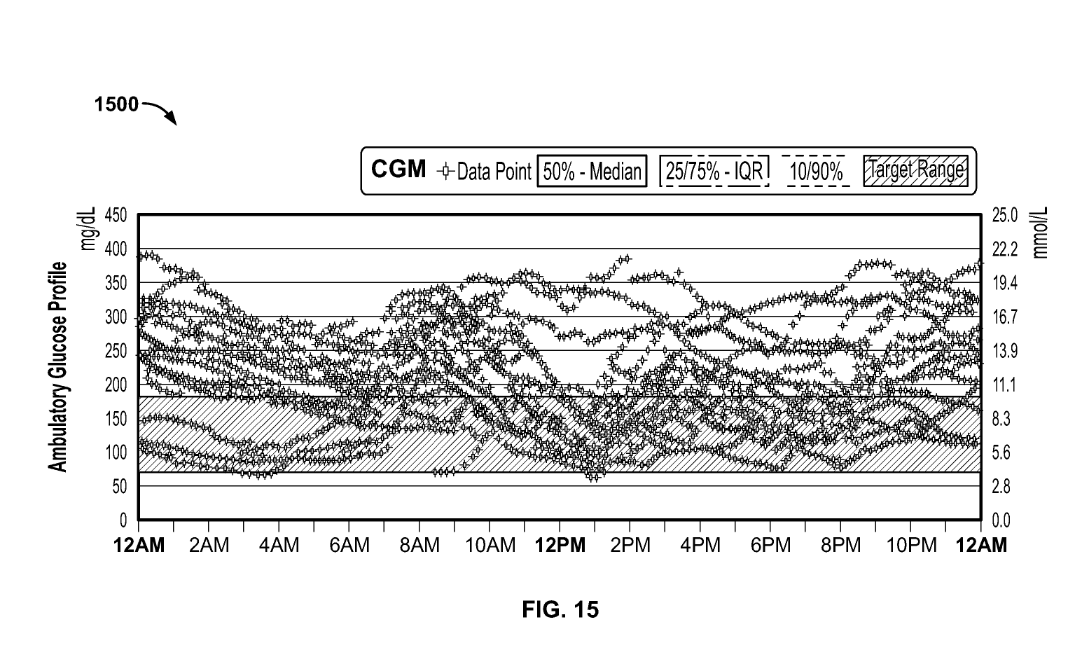

In some embodiments, the daily glucose profile engine 610 calculates and displays the patient's P ambulatory glucose profile in a daily calendar view format. The daily glucose profile window is described in more detail with reference to FIG. 15.

FIG. 7 is an example diagram illustrating a glucose statistics window 700 of the glucose data display described in FIG. 5. In this embodiment, the glucose statistics window 700 displays four categories of glucose statistics labeled glucose exposure 702, glucose variability 704, glucose ranges 706, and data sufficiency 708. The statistics calculated in each of the four categories are based on blood glucose measurements taken by a CGM device or a SMBG device over a given period of time. In some embodiments, the given time period is a 24-hour time period. In other embodiments, other time periods are used.

In this example embodiment, the glucose statistics window 700 is displays the title “Glucose Statistics” 710 vertically on the left edge of the statistics window 700. In other embodiments, the title “Glucose Statistics” 710 is displayed in other areas of the window 700, such as, but not limited to, the top center of the window 700, the bottom center of the window 700, or vertically on the right edge of the window 700. In this example embodiment, each of the four categories includes a label, units of measurement, and a reference range corresponding to a normal range.

In some embodiments, glucose data is determined only by data received from a CGM device. In other embodiments, glucose data is determined only by data received from a SMBG device.

The glucose exposure 702 statistic includes two columns labeled average glucose 712 and estimated HbAlc 714 (as a %). In this embodiment, the unit of measurement for average glucose 712 is expressed in mg/dL. In other embodiments, the unit of measurement for average glucose 712 is expressed in mmol/L.

Average glucose 712 is found by taking the sum of all glucose measurements in a given period and dividing it by the total number of glucose measurements in that given period. The average glucose 712 statistic includes an average glucose reference range 716. In this example embodiment, the average glucose reference range 716 is between 88-116 mg/L or 4.8-6.4 mmol/L. In some embodiments, other reference ranges are used.

HbAlc is commonly known as glycated hemoglobin and refers to the average plasma glucose concentration in the patient's P blood. In some embodiment, the estimated HbAlc 714 is calculated by adding the average glucose 712 with 46.7 and dividing that number by 28.7. In this example embodiment, the estimated HbAlc 714 includes a HbAlc reference range 718 of less than 6. In other embodiments, another reference range is used.

The glucose variability 704 statistic is divided into two columns labeled standard deviation of the glucose measurements 720 and interquartile range (IQR) 722. The standard deviation of the glucose measurements 720 and IQR 722 are expressed in mg/dL. In other embodiments, the standard deviation of the glucose measurements 720 and IQR 722 are expressed in mmol/L.

The standard deviation of glucose measurements 720 is found by the following formula:

$$Standard deviation = \sqrt{\frac{{\sum }_{i = 0}^{N - 1} {( {g}_{i} - \overline{g} )}^{2}}{N}} ,$$

where gᵢ represents a first glucose measurement, N represents the total number of glucose measurements, and g is the average glucose 712. The standard deviation of glucose measurements 720 includes a standard deviation of glucose measurements reference range 724. In this example embodiment, the standard deviation of glucose measurements reference range 724 is 10-26 mg/dL. In other embodiments, other reference ranges and/or units are used.

IQR is commonly known as a measure of statistical dispersion. The IQR 722 is found by calculating the difference between the 75^{th} percentile average and the 25^{th} percentile average. The percentile for a blood glucose value is found by determining the percent (10%, 25%, 50%, 75%, and/or 90%) of all blood glucose levels that fall below the given blood glucose value. Each glucose value, taken by a CGM device or a SMBG device, is placed into one of 24 hourly bins corresponding to the respective time of measurement. Once percentile statistics are calculated for each hourly bin by a computing device, hourly plot points are smoothed using a weighted algorithm that incorporates the previous and following hourly bin values with the target bin. Smoothing generally refers to an approximating algorithm used to capture important data values. In some embodiments, 10^{th}, 25^{th}, median, 75^{th}, and 90^{th} percentiles are calculated for each bin. In other embodiments, other percentiles are calculated. In this example embodiment, the IQR 714 also has a reference range 726 of less than 13-29 mg/dL. In other embodiments, other reference ranges and/or units are used. In some embodiments, if one or more of the hourly bins contains no data, the IQR 714 displays ‘NA’, ‘not applicable’, ‘N/A’ and the like.

In this embodiment, the glucose ranges 706 statistic is divided into seven columns depicting seven glucose ranges, expressed in mg/dL, labeled dangerously low 728, very low 730, low 732, in target 734, high 736, very high 738, and dangerously high 740. In some embodiments, more or less ranges are shown. In other embodiments, the glucose ranges 706 are expressed in mmol/L. The glucose ranges 706 statistic calculates the percentage of a patient's P blood glucose measurements that fall in each range in a given time period.

The default ranges for the following categories are as follows: dangerously low 728 is below 50 mg/dL; very low 730 is below 60 mg/dL; low 732 is below 70 mg/dL; in target 734 is 70-180 mg/dL; high 736 is above 180 mg/dL; very high 738 is above 250 mg/dL; and dangerously high 740 is above 400 mg/dL.

In this example embodiment, the ranges each have reference ranges. In this embodiment, the dangerously low reference range 742 and the very low reference range 744 are set at zero. The low reference range 746 is set at less than 4. The in target reference range 748 is set to greater than 90. The high reference range 750 is set to less than 6; the very high reference range 752 and the dangerously high reference range 754 are set at zero. In other embodiments, other reference ranges are used.

The data sufficiency 708 statistic displays the average tests per day and in some embodiments, it is calculated by dividing the total number of measurements in the current set divided by the number of days measured from the date and time of the first measurement to the date and time of the second measurement. The data sufficiency reference range 756 is depended upon the measurement interval of the device used to obtain the data. For CGM devices with a 10 minute interval, the reference range is maximum 144. For CGM devices with a five minute interval, the reference range is maximum 288. For SMBG devices, there is no reference range and the data sufficiency reference range 756 displays ‘NA’, ‘not applicable’, ‘N/A’ and the like.

FIG. 8 is an example diagram illustrating an optional expanded glucose statistics window 800 displayed below the glucose statistics window 700 illustrated and described with respect to FIG. 7. The expanded glucose statistics window 800 displays the four categories of glucose statistics (standard statistics) as illustrated and described with reference to FIG. 7 and displays three additional categories of glucose statistics (expanded statistics). These expanded statistics are labeled glucose exposure close-up 802, variability close-up 804, and hypoglycemia and hyperglycemia episodes close-up 806. These expanded statistics are calculated and displayed based on CGM data when the standard statistics are CGM device based and are calculated and based on SMBG data when the standard statistics are SMBG device based.

In this example embodiment, the expanded statistics are shown below the standard statistics. In some embodiments, the expanded statistics are placed above the standard statistics. In other embodiments, the expanded statistics are distributed between the standard statistics.

In this embodiment, the glucose exposure close up 802 statistics is divided into three columns labeled wake 808, sleep 810, and 24 hours 812. The wake 808 column is labeled with the patient's P waking hours in the form of HH {AM/PM} to HH {AM/PM}, wherein the first hour listed is the patient's P first wake hour and the second hour listed is the first sleep hour. In this embodiment, the AUC wake reference range 842 is 89-121 (mg/dL)*h. In other embodiments, other reference ranges are used.

In this embodiment, the sleep 810 column is labeled with the patient's P sleeping hours in the form of HH {AM/PM} to HH {AM/PM}, wherein the first hour listed is the patient's P first sleep hour and the second hour listed is the first wake hour. The default wake time is 6 AM and the default sleep time is 12 AM. In this embodiment, the AUC sleep reference range 844 is 85-109 (mg/dL)*h. In other embodiments, other reference ranges are used.

In this embodiment, AUC 24 hours reference range 846 for the 24 hours 812 column is 89-113 (mg/dL)*h. In other embodiments, other reference ranges are used.

In this embodiment, the glucose exposure close-up 802 statistic has an hourly area under the curve (AUC/Hourly) row 814 with a unit of measurement of (mg/dL)*hr. In some embodiments, the AUC/Hourly row 814 has a unit of measurement of (mmol/L)*hr. The AUC/Hourly row 814 is calculated for the wake 808, sleep 810, and 24 hours 812 columns.

The AUC/Hourly row 814 is the total area under the curve divided by 24. The AUC is calculated as a discrete approximation of the area under the smoothed median (50^{th} percentile) curve utilizing a modified rectangle method. In some embodiments, the AUC is calculated as follows:

$$AUC = {\sum }_{i = 0}^{23} {P}_{{s}_{i}}$$

Where:

l is the hour of the day

P_{S}_{ᵢ} the smoothed percentile value for the i^{th} how of the day

In this embodiment, the variability close-up 804 statistic is divided into two columns labeled coefficient of variation 816 and average change in the median curve 818. The CV 816 is derived by the following formula: |(Standard Deviation/Mean)|*100. In this embodiment, the unit of measurement for the coefficient of variation (CV) 816 is a percent. In this embodiment, the CV reference range 820 is 19-25. In other embodiments, other ranges are used. The CV tracks changes in the patient's overall glycemic variability.

The average change in the median curve 818 is derived from the following formula:

$${Δ}_{MC} = \frac{( \vert {g}_{0} - {g}_{23} \vert ) + {\sum }_{t = 1}^{23} \vert {g}_{t} - {g}_{t - 1} \vert}{T}$$

Where:

Δ_{MC} is the Change in the Median Curve

t is an hour of the day (0-23)

g is the smoothed median value for the given hour of the day

T is the total number of non-missing hourly smoothed percentiles

In this embodiment, the unit of measurement for the average change in the median curve 818 is in mg/dL/hr. In other embodiments, the unit of measurement is mmol/L/hr. The average change in the median curve reference range 822 is 2-5. In other embodiments, other ranges are used.

In this embodiment, the hypoglycemia and hyperglycemia episodes close-up 806 tracks how much time the patient spends below the target range, within the target range, or above the target range. In some embodiments the target range and/or percentiles are shaded with one or more colors, and in some embodiments they are each colored with different colors. In this embodiment, the hypoglycemia and hyperglycemia episodes close-up 806 is split into six columns representing ranges and three rows. The six columns of episode ranges are labeled <50 824, <60 826, <70 828, >180 830, >250 832, and >400 834. The measurement unit for each range is in mg/dL. In other embodiments, mmol/L is used. In this embodiment, the below threshold range is 50-60 mg/dL; the target threshold range is 70-180 mg/dL; and the above threshold range is 250-400 mg/dL. In this embodiment, these ranges are the default settings and can be adjusted to other settings as defined by the user.

In this embodiment, the three rows are labeled average hours per day 836, mean episodes per day 838, and mean duration (hours) 840. In this embodiment, an episode is defined as at least ten minutes of consecutive measurements within a range, thus once the reads are below or above a target and last for ten minutes, the episode continues until a reading moves up or down into a new target range.

In this embodiment, the average hours per day 836 is calculated differently for each threshold range. In some embodiments, the average hours per day 836 of episodes below threshold (i.e. between 50-60 mg/dL) is derived from the following formula:

H̅_̅{̅T̅}̅_̅{̅ₗ̅}̅= E̅_̅{̅T̅}̅_̅{̅ₗ̅}̅× D̅_̅{̅T̅}̅_̅{̅ₗ̅}̅

Where:

H_{T}_{ₗ} is the average hours per day spent in episodes below the lower threshold

E̅_̅{̅T̅}̅_̅{̅ₗ̅}̅ is the mean episodes below the lower threshold per day

D̅_̅{̅T̅}̅_̅{̅ₗ̅}̅ is the mean duration of episodes below the lower threshold

In some embodiments, the average hours per day 836 of episodes in the within threshold (i.e. between 70-180 mg/dL) is derived from the following formula:

H̅_̅{̅T̅}̅_̅{̅_̅{̅w̅}̅}̅= E̅_̅{̅T̅}̅_̅{̅_̅{̅w̅}̅}̅× D̅_̅{̅T̅}̅_̅{̅_̅{̅w̅}̅}̅

Where:

H_{T}_{_{w}} is the average hours per day spent in episodes within the lower and upper thresholds

E̅_̅{̅T̅}̅_̅{̅_̅{̅w̅}̅}̅ is the mean episodes within the lower and upper thresholds per day

D̅_̅{̅T̅}̅_̅{̅_̅{̅w̅}̅}̅ is the mean duration of episodes within the lower and upper thresholds

In some embodiments, the average hours per day 836 of episodes above threshold (i.e. between 250-400 mg/dL) is derived from the following formula:

H̅_̅{̅T̅}̅_̅{̅ᵤ̅}̅= E̅_̅{̅T̅}̅_̅{̅ₕ̅}̅× D̅_̅{̅T̅}̅_̅{̅ᵤ̅}̅

H_{T}_{ᵤ} is the average hours per day spent in episodes above the upper threshold

E̅_̅{̅T̅}̅_̅{̅ₕ̅}̅ is the mean episodes above the upper threshold per day

D̅_̅{̅T̅}̅_̅{̅ᵤ̅}̅ is the mean duration of episodes above the upper threshold

The standard display for this value is rounded to one place after the decimal point.

In this embodiment, the mean episodes per day 838 is calculated differently for each threshold range. In some embodiments, the mean episodes per day 838 of episodes below threshold (i.e. between 50-60 mg/dL) is derived from the following formula:

$$\overline{{E}_{{T}_{l}}} = \vert {E}_{{T}_{l}} \vert \times ( \frac{1440}{I} ) / \vert {g}_{all} \vert$$

Where:

E̅_̅{̅T̅}̅_̅{̅ᵢ̅}̅ is the mean episodes below threshold per day

|E_{T}_{ᵢ}| is the cardinality of all episodes below the lower threshold

l is the meter measurement interval in minutes

|gₐₗₗ| is the cardinality of all measurements not discarded

In some embodiments, the mean episodes per day 838 of episodes within threshold (i.e. between 70-180 mg/dL) is derived from the following formula:

$$\overline{{E}_{{T}_{w}}} = \vert {E}_{{T}_{w}} \vert \times ( \frac{1440}{I} ) / \vert {g}_{all} \vert$$

Where:

E̅_̅{̅T̅}̅_̅{̅_̅{̅w̅}̅}̅ is the mean episodes within the lower and upper threshold per day

|E_{T}_{_{w}}| is the cardinality of all episodes within the lower and upper thresholds

l is the meter measurement interval in minutes

|gₐₗₗ| is the cardinality of all measurements not discarded

In some embodiments, the mean episodes per day 838 of episodes above threshold (i.e. between 250-400 mg/dL) is derived from the following formula:

$$\overline{{E}_{{T}_{h}}} = \vert {E}_{{T}_{h}} \vert \times ( \frac{1440}{I} ) / \vert {g}_{all} \vert$$

### Where:

E̅_̅{̅T̅}̅_̅{̅ₕ̅}̅ the mean episodes above the upper threshold per day

|E_{T}_{ₕ}| is the cardinality of all episodes above the upper threshold

l is the meter measurement interval in minutes

|gₐₗₗ| is the cardinality of all measurements not discarded

FIG. 9 is an example diagram illustrating an ambulatory glucose profile (AGP) window 900 of the glucose data display 500 described in FIG. 5. The AGP window 900 helps illustrate the patient's P glucose pattern over a 24-hour period using a CGM device. The AGP window 900 includes a graph 902 with an x-axis 904, a left y-axis 906, a right y-axis 908, a target range 910, a 10%-90% percentile range 912, a 25%-75% percentile range (IQR) 914, and a median line 916. In this embodiment, the AGP window 900 also includes a legend 918 describing the various lines and points displayed on the graph 902. In some embodiments, the viewer can choose to view CGM device data points individually on the graph 902.

In this embodiment, the graph 902 presents a modal day, or standard 24-hour day, visual display of the patient's collected glucose data. In this embodiment, the x-axis 904 represents time, in hours, and starts at 12 AM and ends at 12 PM, with hash marks representing every hour. Additionally, in this embodiment a time label every two hours is displayed on the x-axis 904. In other embodiments, more or less time labels are used. Yet in other embodiments, astronomical time is displayed on the x-axis 904, starting at 00:00 and ending at 24:00.

In this embodiment, the left and right y-axes 906 and 908, respectively, represent blood glucose values. In this embodiment, the left y-axis 906 has units of measurement in mg/dL whereas the right y-axis 906 has units of measurement in mmol/L. In other embodiments, the units of measurements are switched, and yet in other embodiments, other units of measurement are used. In this embodiment, a horizontal line crossing the left y-axis 906 to the right y-axis 908 is displayed every 50 mg/dL. In some embodiments, more or less horizontal lines are shown. Yet in other embodiments, no horizontal lines are shown.

In this embodiment, a target range 910 is shown. The target range is bounded by upper and lower boundary lines 920 and 922, respectively, wherein the default lower boundary line 922 is set at 70 mg/dL and the default upper boundary line 924 is set at 180 mg/dL. In this embodiment, default ranges can be changed. In other embodiments, other default target ranges are set.

In this embodiment, the graph 902 displays data from the patient's P CGM device. In this embodiment, at every 60 minute interval, if at least one CGM glucose measurement is available, the median of the glucose measurements is calculated and smoothed. Additionally, in this embodiment, all smoothed median CGM values within one hour of each other are connected by a line indicated by the median line 916.

In this embodiment, at every 60 minute interval, if at least one CGM glucose measurement is available, the 75^{th} percentile measurement is calculated and smoothed. Additionally, in this embodiment, all smoothed 75^{th} percentile measurements within one hour of each other are connected by a line indicated by the 75^{th} percentile line 924.

Also in this embodiment, at every 60 minute interval, if at least one CGM glucose measurement is available, the 25^{th} percentile measurement is calculated and smoothed. Additionally, in this embodiment, all smoothed 25^{th} percentile measurements within one hour of each other are connected by a line indicated by the 25^{th} percentile line 926.

Also in this embodiment, at every 60 minute interval, if at least one CGM glucose measurement is available, the 90^{th} percentile measurement is calculated and smoothed. Additionally, in this embodiment, all smoothed 90^{th} percentile measurements within one hour of each other are connected by a line indicated by the 90^{th} percentile line 928.

Also in this embodiment, at every 60 minute interval, if at least one CGM glucose measurement is available, the 10^{th} percentile measurement is calculated and smoothed. Additionally, in this embodiment, all smoothed 10^{th} percentile measurements within one hour of each other are connected by a line indicated by the 10^{th} percentile line 930.

In some embodiments, individual CGM device data points are shown on the graph 902. In other embodiments, individual SMBG device data points are shown on the graph 902.

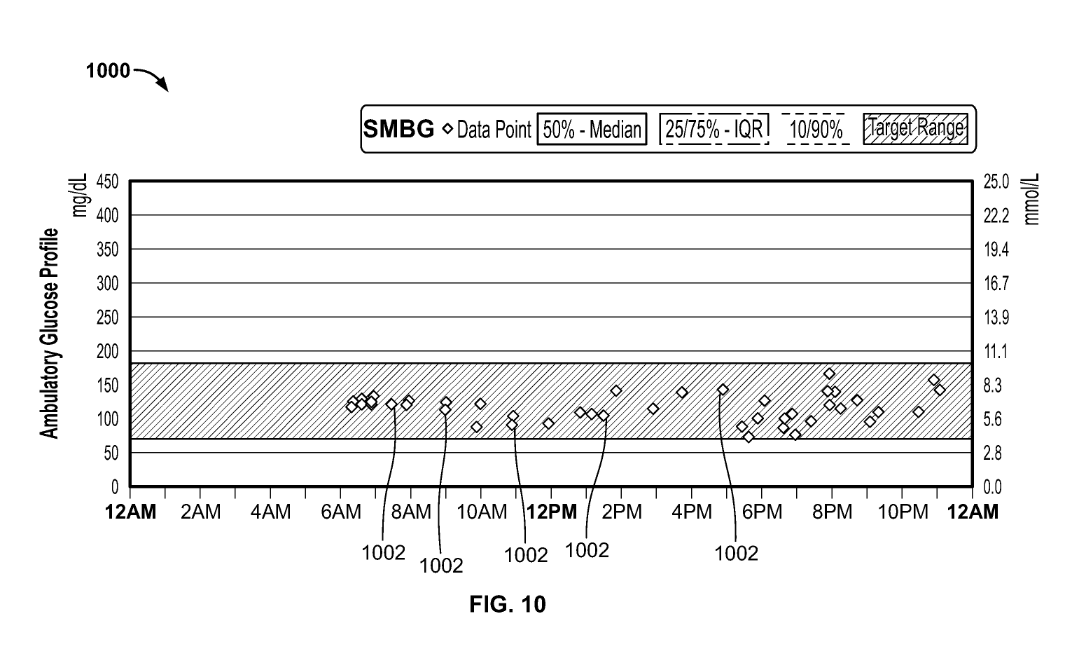

FIG. 10 is an alternative example diagram illustrating an AGP window 1000 of the glucose data display described in FIGS. 5 and 9. Like the AGP window 900 in FIG. 9, this alternative embodiment contains a graph 902 with an x-axis 904, a left y-axis 906, a right y-axis 908, and a target range 910. In this embodiment, the AGP window 900 also includes a legend 918 describing the various lines and points displayed on the graph 902. This alternative embodiment displays only SMBG device data points 1002. In some embodiments, if no CGM device measurements are taken, then only SMBG device data points 1002 are illustrated on the graph 902. In some embodiments, both data points are shown using different data points if both CGM device measurements and SMBG device measurements are taken. In other embodiments, the viewer can decide which device measurements to display if both CGM device measurements and SMBG device measurements are taken.

In this embodiment, the graph 902 displays data from the patient's P SMBG device. In this embodiment, at every 60 minute interval, if at least one SMBG glucose measurement is available, the median of the glucose measurements is calculated and smoothed. Additionally, in this embodiment, all smoothed median SMBG values within one hour of each other are connected by a line.

In this embodiment, at every 60 minute interval, if at least one SMBG glucose measurement is available, the 75^{th} percentile measurement is calculated and smoothed. Additionally, in this embodiment, all smoothed 75^{th} percentile measurements within one hour of each other are connected by a line.

Also in this embodiment, at every 60 minute interval, if at least one SMBG glucose measurement is available, the 25^{th} percentile measurement is calculated and smoothed. Additionally, in this embodiment, all smoothed 25^{th} percentile measurements within one hour of each other are connected by a line.

Also in this embodiment, at every 60 minute interval, if at least one SMBG glucose measurement is available, the 90^{th} percentile measurement is calculated and smoothed. Additionally, in this embodiment, all smoothed 90^{th} percentile measurements within one hour of each other are connected by a line.

Also in this embodiment, at every 60 minute interval, if at least one SMBG glucose measurement is available, the 10^{th} percentile measurement is calculated and smoothed. Additionally, in this embodiment, all smoothed 10^{th} percentile measurements within one hour of each other are connected by a line.

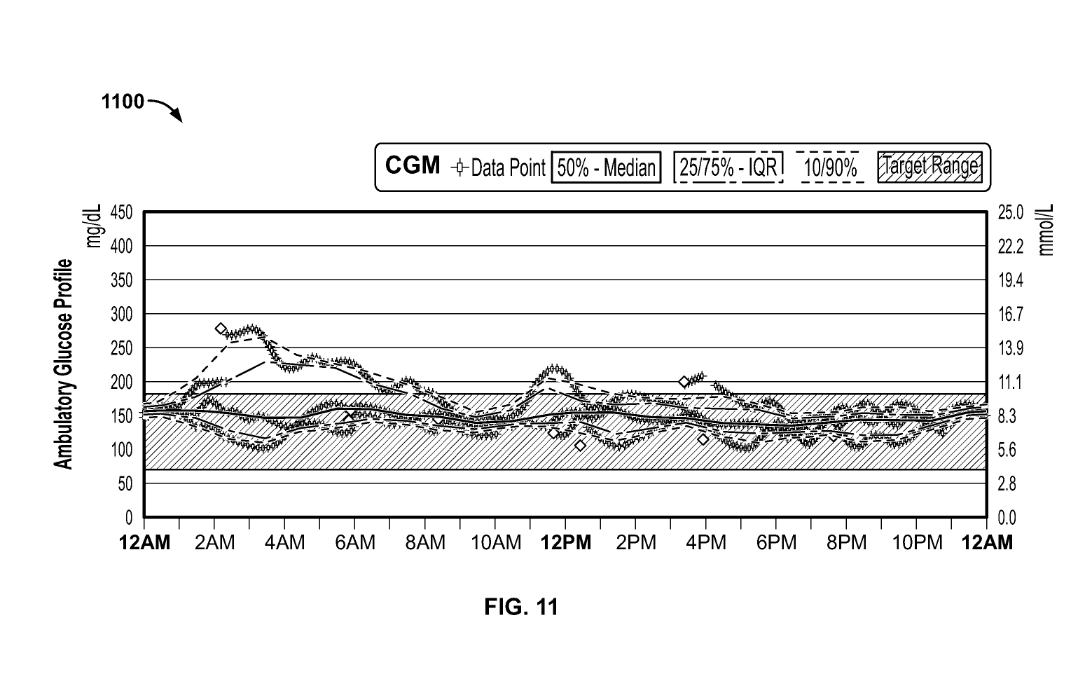

FIGS. 11-15 illustrate other example embodiments of the AGP window 504. FIG. 11 illustrates an example embodiment of an AGP window 1100 including CGM data lines, CGM data points, and SMBG data points.

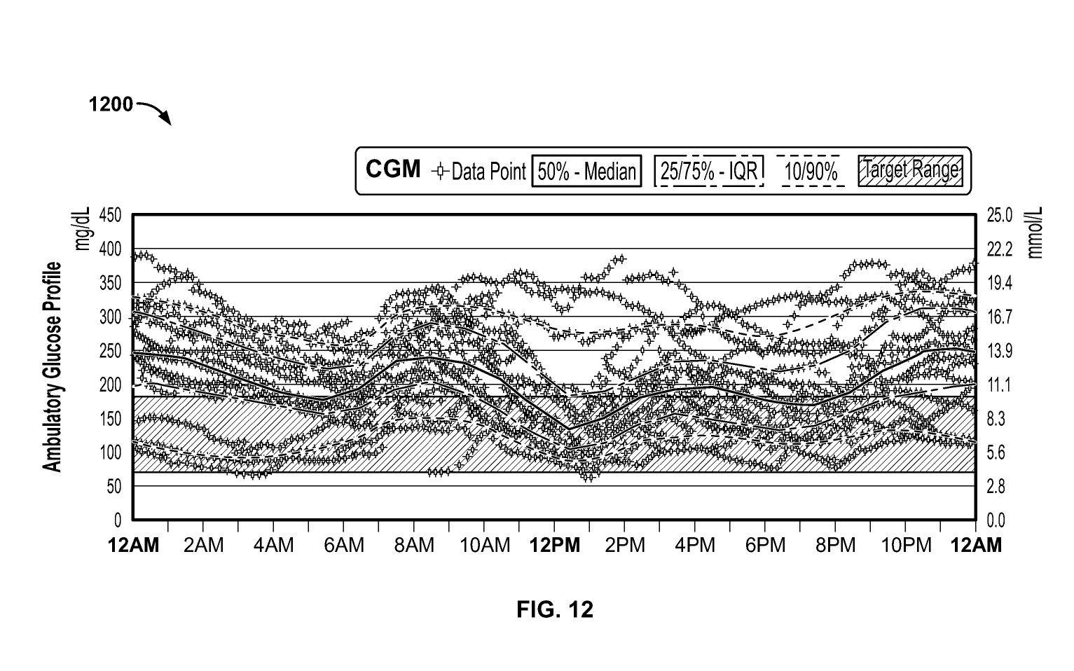

FIG. 12 is another example embodiment of an AGP window 1200 including CGM data lines and CGM data points.

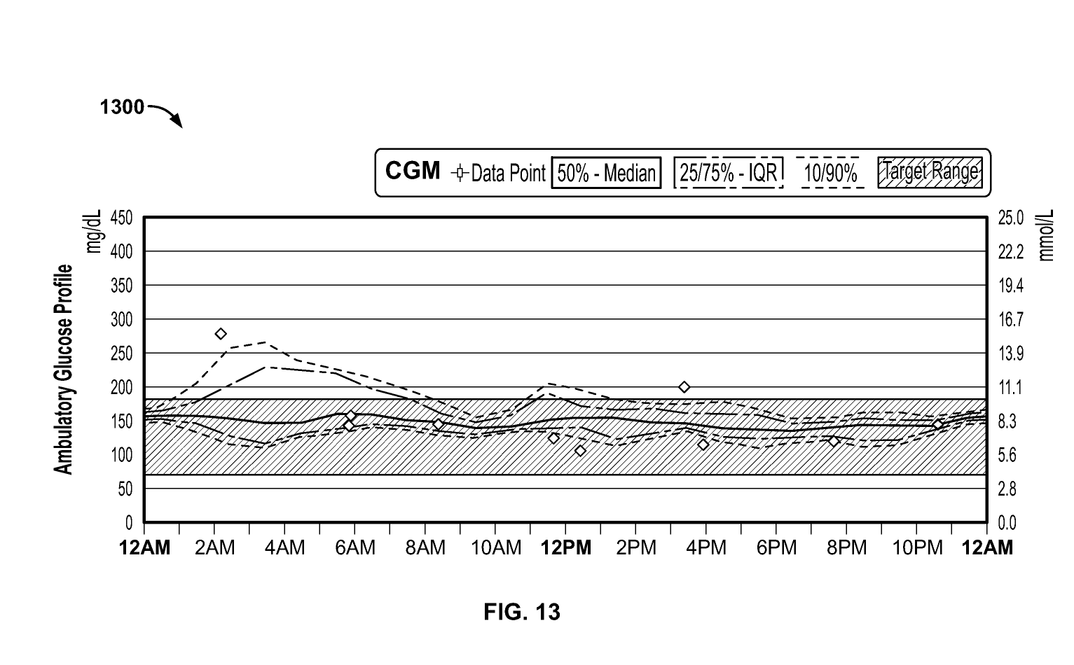

FIG. 13 is another example embodiment of an AGP window 1300 including CGM data lines and SMBG data points.

FIG. 14 is another example embodiment of an AGP window 1400 including CGM data points and SMBG data points.

FIG. 15 is another example embodiment of an AGP window 1500 including only CGM data points.

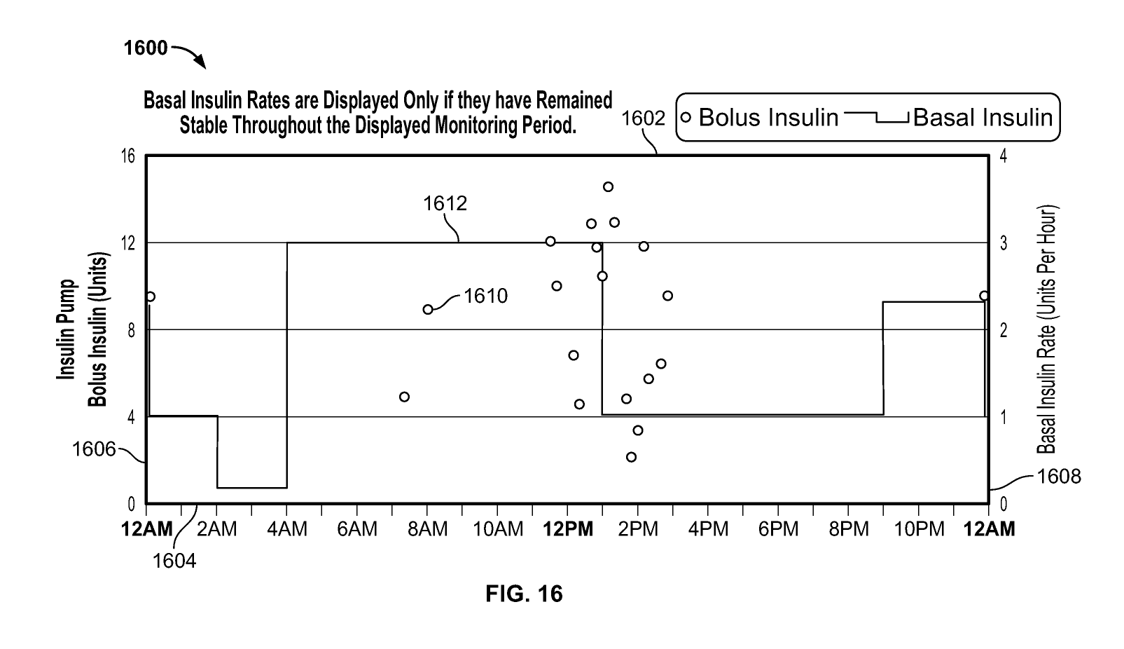

FIG. 16 is an example diagram illustrating an insulin pump graph window 1600 of the glucose data display 500 described in FIG. 5. In some embodiments, the insulin pump graph window 1600 is displayed below the AGP window 504. In other embodiments, the insulin pump graph window 1600 is not displayed.

In this embodiment, the insulin pump graph window 1600 includes a graph 1602 with an x-axis 1604, a left y-axis 1606, a right y-axis 1608, and a legend 1610.

In this embodiment, the graph 1602 represents a modal day visual display of the patient's collected insulin data. In this embodiment, the x-axis 1604 represents time, in hours and starts at 12 AM and ends at 12 PM with hash marks representing each hour. Additionally, in this embodiment, a time label every two hours is displayed on the x-axis 1604. In other embodiments, more or less time labels are used. Yet in other embodiments, astronomical time is displayed on the x-axis 1604, starting at 00:00 and ending at 24:00. In this embodiment, the insulin pump graph 1602 is has a width consistent with the AGP graph as illustrated and described in FIGS. 9-15.

In this embodiment, the left and right y-axes 1606 and 1608, respectively represent insulin levels. In this embodiment, the left y-axis 1606 represents the patient's P bolus insulin that is measured in units. In this embodiment, the left y-axis 1606 starts at 0 units and ends at 16 units. In this embodiment, the bolus insulin levels are displayed as data points 1610.

Also in this embodiment, the right y-axis 1608 represents the patient's basal insulin that is measured in units per hour. In this embodiment, the right y-axis 1608 starts at 0 units per hour and ends at 4 units per hour. In this embodiment, the basal insulin rates are displayed only if they have remained stable throughout the displayed monitoring period. In this embodiment, the basal insulin data points are displayed as a stepped line 1612.

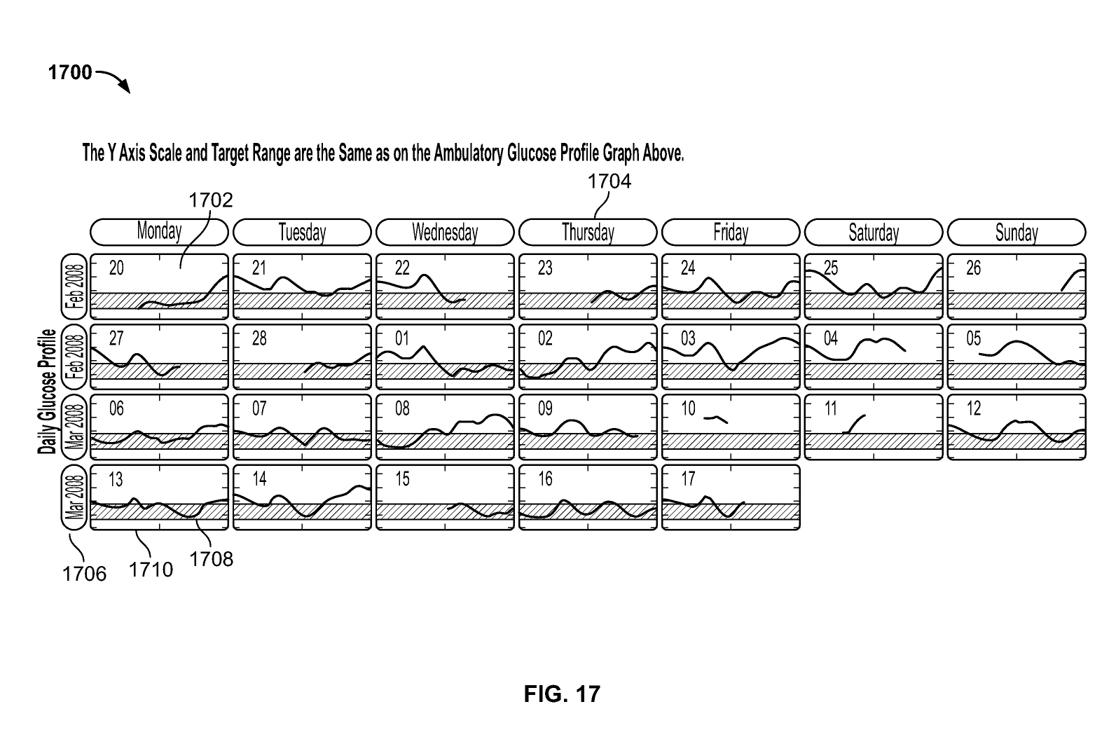

FIG. 17 is an example diagram illustrating a daily glucose profile window 1700 of the glucose data display 500 described in FIG. 5. In this embodiment, the daily glucose profile window 1700 includes thumbnails 1702 arranged in a calendar format, an x-axis 1704, and a y-axis 1706. In this embodiment, the x-axis 1704 displays the days of the week starting with Monday and ending with Sunday. In other embodiments, other start and end days are used. In this embodiment, the y-axis 1706 displays the month and year, thereby displaying the daily glucose profile window 1700 as a monthly calendar. In this embodiment, the daily glucose profile window 1700 displays four rows of data. In other embodiments, daily glucose profile window 1700 displays more or less rows of data.

In this embodiment, the thumbnails 1702 include an x-axis 1708 and a y-axis 1710 that correspond to the x-axis and y-axis of the AGP window 504 as described and illustrated in FIGS. 9-15. Additionally, each thumbnail 1702 includes a date corresponding to the day of the month from which the glucose data was derived. In this embodiment, each thumbnail also includes a target range 1712 and a median line 1714 corresponding to the target range and the median line in the AGP window 504. Also in this embodiment, selecting a thumbnail 1702 for a given day will expand it to display the AGP window 504 for that selected day.

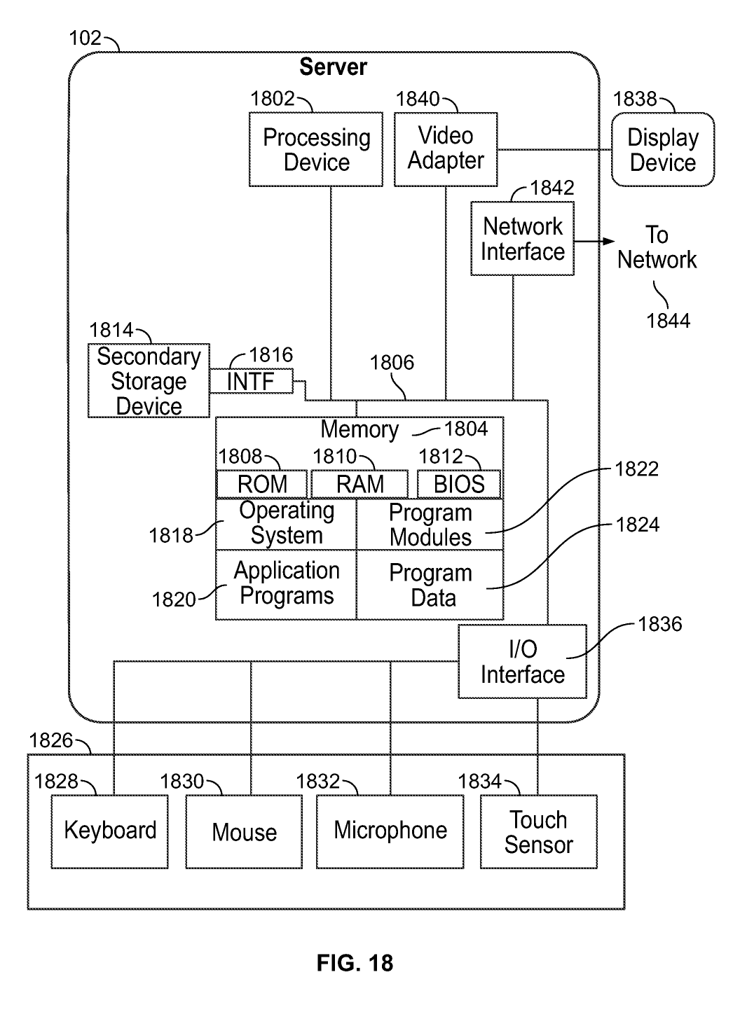

FIG. 18 illustrates an exemplary architecture of a computing device that can be used to implement aspects of the present disclosure. The computing device illustrated in FIG. 1 can be used to execute the operating system, application programs, and software modules (including the software engines) described herein.

The computing device 102 includes, in some embodiments, at least one processing device 1802, such as a central processing unit (CPU). A variety of processing devices are available from a variety of manufacturers, for example, Intel or Advanced Micro Devices. In this example, the computing device 102 also includes a system memory 1804, and a system bus 1806 that couples various system components including the system memory 1804 to the processing device 1802. The system bus 1806 is one of any number of types of bus structures including a memory bus, or memory controller; a peripheral bus; and a local bus using any of a variety of bus architectures.

Examples of computing devices suitable for the computing device 102 include a desktop computer, a laptop computer, a tablet computer, a mobile computing device (such as a smart phone, an iPod® or iPad® mobile digital device, or other mobile devices), or other devices configured to process digital instructions.

The system memory 1804 includes read only memory 1808 and random access memory 1810. A basic input/output system 1812 containing the basic routines that act to transfer information within computing device 102, such as during start up, is typically stored in the read only memory 1808.

The computing device 102 also includes a secondary storage device 1814 in some embodiments, such as a hard disk drive, for storing digital data. The secondary storage device 1814 is connected to the system bus 1806 by a secondary storage interface 1816. The secondary storage devices 1814 and their associated computer readable media provide nonvolatile storage of computer readable instructions (including application programs and program modules), data structures, and other data for the computing device 102.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. Additionally, such computer readable storage media can include local storage or cloud-based storage.

A number of program modules can be stored in secondary storage device 1814 or memory 1804, including an operating system 1818, one or more application programs 1820, other program modules 1822 (such as the software engines described herein), and program data 1824. The computing device 102 can utilize any suitable operating system, such as Microsoft Windows™, Google Chrome™, Apple OS, and any other operating system suitable for a computing device.

In some embodiments, a user provides inputs to the computing device 102 through one or more input devices 1826. Examples of input devices 1826 include a keyboard 1828, mouse 1830, microphone 1832, and touch sensor 1834 (such as a touchpad or touch sensitive display). Other embodiments include other input devices 1826. The input devices are often connected to the processing device 1802 through an input/output interface 1836 that is coupled to the system bus 1806. These input devices 1826 can be connected by any number of input/output interfaces, such as a parallel port, serial port, game port, or a universal serial bus. Wireless communication between input devices and the interface 1836 is possible as well, and includes infrared, BLUETOOTH® wireless technology, 802.11a/b/g/n, cellular, or other radio frequency communication systems in some possible embodiments.

In this example embodiment, a display device 1838, such as a monitor, liquid crystal display device, projector, or touch sensitive display device, is also connected to the system bus 1806 via an interface, such as a video adapter 1840. In addition to the display device 1838, the computing device 102 can include various other peripheral devices (not shown), such as speakers or a printer.

When used in a local area networking environment or a wide area networking environment (such as the Internet), the computing device 102 is typically connected to the network 1844 through a network interface 1842 as an Ethernet interface. Other possible embodiments use other communication devices. For example, some embodiments of the computing device 102 include a modem for communicating across the network.

The computing device 102 typically includes at least some form of computer readable media. Computer readable media includes any available media that can be accessed by the computing device 102. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 102. Computer readable storage media does not include computer readable communication media.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 18 is also an example of programmable electronics, which may include one or more such computing devices, and when multiple computing devices are included, such computing devices can be coupled together with a suitable data communication network so as to collectively perform the various functions, methods, or operations disclosed herein.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A method of evaluating and displaying glucose data, the method comprising:
receiving at a computing device glucose data for a patient, the glucose data containing data generated by a continuous glucose monitor associated with the patient; and
generating a graphical display of the glucose data with the computing device, the graphical display including at least a glucose profile for a modal day, the glucose profile graphically depicting therein:
a target range for the glucose data for the patient, including at least an upper boundary and a lower boundary; and
a line representing a median value of the glucose data across the modal day.

2. The method of claim 1, wherein the glucose profile further displays lines graphically depicting a first lower boundary of the data points and a first upper boundary of the data points.

3. The method of claim 2, wherein the first lower boundary depicts the 10 percentile boundary or the 25 percentile boundary, and wherein the first upper boundary depicts the 90 percentile boundary or the 75 percentile boundary.

4. The method of claim 2, wherein the glucose profile further displays lines graphically depicting a lower intermediate boundary of the data points an upper intermediate boundary of the data points.

5. The method of claim 4, wherein the lower intermediate boundary is the 25 percentile boundary, and wherein the upper intermediate boundary is the 75 percentile boundary.

6. The method of claim 1, wherein the glucose profile further displays data points representing glucose data collected by a self-monitoring blood glucose device.

7. The method of claim 4, wherein the line is a continuous line, and wherein the line is displayed in a first color, the first color being different than all other colors displayed in the graphical display.

8. The method of claim 7, wherein the target range is shaded with a second color in the graphical display.

9. The method of claim 8, wherein a range of data points between the lower intermediate boundary and the upper intermediate boundary are shaded with a third color in the graphical display.

10. The method of claim 1, further comprising transmitting the graphical display to a remote computing device for visual presentation to a caregiver.

11. A method of graphically displaying glucose data, the method comprising:
evaluating glucose data, the glucose data including data obtained from a glucose monitor device;
generating with a computing device a glucose statistics window based on the evaluation of the glucose data, the glucose statistics window including at least a glucose exposure statistic, a glucose variability statistic, glucose ranges, and a data sufficiency statistic;
generating an ambulatory glucose profile window, the ambulatory glucose profile window including a graphical display of the glucose data across a modal day; and
generating a daily glucose profile window, the daily glucose profile window including a graphical display of the glucose data corresponding to days of a week.

12. The method of claim 11, further comprising:
generating an insulin pump window, the insulin pump window including a graphical display of insulin data across the modal day.

13. The method of claim 12, wherein the insulin data comprises bolus insulin data and basal insulin data.

14. The method of claim 11, wherein the glucose statistics window further comprises expanded statistics, the expanded statistics including a glucose exposure close-up statistics, variability close-up statistics, and hypoglycemia and hyperglycemia episodes close-up statistics.

15. A glucose data evaluation server, comprising:
a computing device; and
at least one computer readable storage device, the at least one computer readable storage device storing (i) glucose data based at least in part upon data obtained by a continuous glucose monitor device, and (ii) program instructions, the program instructions being executable by the computing device to:
generate a graphical display of the glucose data, the graphical display including at least a glucose profile for a modal day, the glucose profile graphically depicting therein:
a target range for the glucose data for the patient, including at least an upper boundary and a lower boundary; and
a line representing a median value of the glucose data across the modal day.

16. A glucose data evaluation server, comprising:
a computing device; and
at least one computer readable storage device, the at least one computer readable storage device storing (i) glucose data based at least in part upon data obtained by a continuous glucose monitor device, and (ii) program instructions, the program instructions being executable by the computing device to:
generate a glucose statistics window based on the glucose data, the glucose statistics window including at least a glucose exposure statistic, a glucose variability statistic, glucose ranges, and a data sufficiency statistic;
generate an ambulatory glucose profile window, the ambulatory glucose profile window including a graphical display of the glucose data across a modal day; and
generate a daily glucose profile window, the daily glucose profile window including a graphical display of the glucose data corresponding to days of a week.
